(19) United States
(12) Patent Application Publication
(10) Pub. No.: US 2008/0047822 A1
(43) Pub. Date: Feb. 28, 2008

(54) System and method for providing ethanol dehydration services

(76) Inventor: Michael R. Ling, Alpharetta, GA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/584,902

(22) Filed: Oct. 23, 2006

Related U.S. Application Data
(60) Provisional application No. 60/840,111, filed on Aug. 25, 2006.

(51) Int. Cl. C07C 29/80

(52) U.S. Cl. 203/19

(57) ABSTRACT

A method for providing ethanol dehydration services is disclosed. A farmer may take advantages of a new service through the use of a resident ethanol production facility and produce hydrous alcohol. An ethanol dehydration service provider may set up a small scale ethanol dehydration service facility adjacent to the farmer, or alternatively, pick up the hydrous alcohol from the farmer. The farmer delivers hydrous ethanol to the ethanol dehydration service facility where dehydration service is performed. After dehydration, dehydrated ethanol is returned to the farmer who can then sell it for a better price to fuel blending companies, or blend it into E-85 or other motor fuel.

### REFERENCE TO RELATED APPLICATION

This application claims the benefit of the U.S. Provisional Application No. 60/840,111, System And Method For Providing ethanol Dehydration Services, filed on Aug. 25, 2006, the specification of which is incorporated herein in its entirety by this reference.

### BACKGROUND OF THE INVENTION

1. Field of the Invention

The present invention generally relates to the production of ethanol. Specifically, the present invention relates to a system and method for providing dehydration services to parties having distilled ethanol to give them pure ethanol, particularly for the preparation of an ethanol-gasoline mixture.

2. Description of the Related Art

Ethanol has been produced for thousands of years around the world. Distilled spirits (higher concentrations of ethanol in water) is made from fermenting, boiling, and condensing beer or wine. Ethanol is completely miscible in water, allowing a homogenous solution of 0% to 100% ethanol to water mixture. Ethanol boils off at a lower temperature than water, allowing the ethanol to be concentrated through distillation. However, at a 96% ethanol to water mixture, the ethanol and water will boil at the same temperature (an azeotropic mixture). This 96% concentration is the knee on the curve that distillation cannot overcome when one works to remove all of the water in ethanol.

This is extremely important in the use of ethanol as a motor fuel. Ethanol does not naturally have enough vapor pressure to operate properly as a fuel in cars in cooler climates. A portion of gasoline is needed in most cars to allow cold starting. In the United States a mixture of 85% ethanol and 15% gasoline (E-85) is used (or more gasoline in winter months) for flexible fuel vehicles to operate properly.

This presents a problem to the use of ethanol, as gasoline can be blended only if there is less than 1% water in the ethanol. The ethanol needs to be nearly completely free of water in order to blend with gasoline (pure alcohol commonly referred to as “absolute”). Should more water be introduced with the mixture, the gasoline and ethanol will separate, forming a layer of gasoline and a layer of ethanol.

The problem of completely removing the water from ethanol has been a challenge for many years. In one common industrial method to obtain 100% pure alcohol, a small quantity of benzene is added to rectified spirit and the mixture is then distilled. Absolute alcohol is obtained in third fraction that distills over at 78.2° C. (351.3 K). Because a small amount of the benzene used remains in the solution, absolute alcohol produced by this method is not suitable for consumption as benzene is carcinogenic. There is also an absolute alcohol production process by desiccation using glycerol. Absolute alcohol produced by this method is known as spectroscopic alcohol.

During the energy crisis of the early 1980s however, it was discovered that beads made up of a ceramic that had a certain molecular structure with billions of 3 Å holes would very aggressively attract water molecules, whose size being just shy of 3 Å in diameter. The ethanol molecule is too large to be attracted by the hole and passes over without interaction. A grouping of the ceramic beads to dehydrate ethanol is typically called a “molecular sieve.”

In dehydration, a large tank of these beads in a molecular sieve removes the remaining 4-5% of the water and provides a commercially viable (and unlike previous methods, environmentally friendly) method to produce dehydrated ethanol. The 3 Å beads, after becoming saturated with water, are regenerated by heating them to high temperature or by exposing them to low pressure, or a combination of both. This regeneration cycle takes time to complete. Current ethanol plants usually have two large molecular sieve vessels. One of the vessels is online while the other regenerates. Due to the requirements of the vessel regarding volume, piping, insulation, corrosion resistance, and negative pressure, the vessels tend to be quite large. To minimize cost, the vessels have a large volume to surface area ratio. Additionally, the vessels are sized to allow a given ethanol plant's capacity to be fully accommodated in two containers. One to be on-line, and the other to be in regeneration. These molecular sieves are expensive but necessary to allow ethanol to be used in the energy market.

Economies of scale of the dehydration plant dictate that the larger the molecular sieve, the more capacity one can get from a given dollar of expense. Otherwise, there would be many small tanks instead of two large tanks.

FIG. 1 illustrates a prior art market process 100 for an ethanol production process. Typically farmers deliver corn, or other agricultural products suitable for ethanol production, to a larger, geographically centralized ethanol processing plant. The corn is ground and fermented and ethanol produced. The ethanol is distilled and can be dehydrated as described above; it can then be distributed to fuel blending facilities 106. The ethanol may also be sold directly in the ethanol market 108. The by-products also can be sold to the by-product market 110. The farmers provide only raw material to the entire process and harvest limited financial benefits.

However, several limitations make modification of the market process 100 difficult. The modification of the market process 100 is affected by equipment design, facility constraints, and the current business model. If one were to go counter to the prevailing equipment design regarding size—to shrink the size down—one would see decreased volume to surface area. This would make material requirements increase versus a given volume of desiccant. The price of the molecular sieve would go down, but the capacity would decrease far faster. It would not make sense to purposefully increase the cost per gallon of water removed.

If one were to go counter to the prevailing equipment design regarding regeneration vessel arrangement—to make only one channel instead of two channels—one would see an interruption of service. Continuous flow systems are more efficient and easy to control instead of batch processes. Without having a continuous channel for the ethanol to be dehydrated, the upstream processes would either need to be shut down or some system would need to be designed to store and control the incoming ethanol. This would add extra expense and make management of work in process more difficult.

One can also deviate from the prevailing equipment design regarding number of dehydration systems—to make multiple systems to dehydrate instead of just one—but one would see a great increase in cost due to the duplication of piping, valves, and controls. It is far less costly to have only one system than two or more complete systems.

The prevailing equipment design dictates the matching of distillation and dehydration capacity. To make the distillation capacity of the plant much smaller than the dehydration capacity of the plant—one would see increased cost relative to overall plant output of dehydrated ethanol.

The prevailing model of building ever more efficient and advanced equipment means that one would not design equipment that is less efficient solely because it is simpler to design, build, or mass produce. To do so would be removing oneself from the income stream of designing and building expensive ethanol plants.

The prevailing business model of controlling all value added steps also is contrary to the possibility of allowing backyard still operators to bring in their home brewed ethanol and dehydrate it for them; they would reduce their overall value added by promoting competition and consume their time by measuring the quality of and processing small batches of home distillation.

Therefore, there is needed a system and method that can overcome the above limitations and it is to such a system and method the present invention is primarily directed.

### SUMMARY OF THE INVENTION

The present invention enables the possibility of allowing small still operators to bring their home brewed ethanol to a nearby dehydration service station and dehydrate the ethanol at the dehydration service station. In one embodiment, there is provided a method for obtaining dehydrated alcohol by farm product producers who produce ethanol-production-capable farm products. The method includes the steps of producing hydrous ethanol, delivering the hydrous ethanol to a dehydration service provider, and receiving dehydrated ethanol from the dehydration service provider. The method may further comprise establishing an ethanol production facility, preparation of the ethanol-production-capable farm products for fermentation, fermenting the prepared ethanol-production-capable farm products, distilling ethanol, and entering a service agreement with the dehydration service provider. The method may also include installing necessary equipment and hiring a third party to operate the equipment.

In another embodiment, there is provided a method for providing ethanol dehydration services to farm product producers who produce hydrous ethanol. The method includes the steps of receiving hydrous ethanol from a farm product producer, dehydrating the hydrous ethanol, and returning dehydrated ethanol to the farm product producer. The method may further comprise entering a service agreement with the farm product producer, producing a blended fuel by blending the dehydrated ethanol with gasoline, and selling the blended fuel.

In yet another alternative embodiment, there is provided a system for dehydrating hydrous ethanol produced by a farmer. The system includes the steps of a farm having an ethanol production facility, and an ethanol dehydration facility adjacent to the farm. The ethanol production facility may further comprise a farm having an ethanol production facility, and an ethanol dehydration facility adjacent to the farm.

Other advantages and features of the present invention will become apparent after review of the hereinafter set forth Description of the Drawings, Detailed Description of the Invention, and the Claims.

### DESCRIPTION OF THE DRAWINGS

FIG. 1 illustrates a prior art market process 100 for ethanol process.

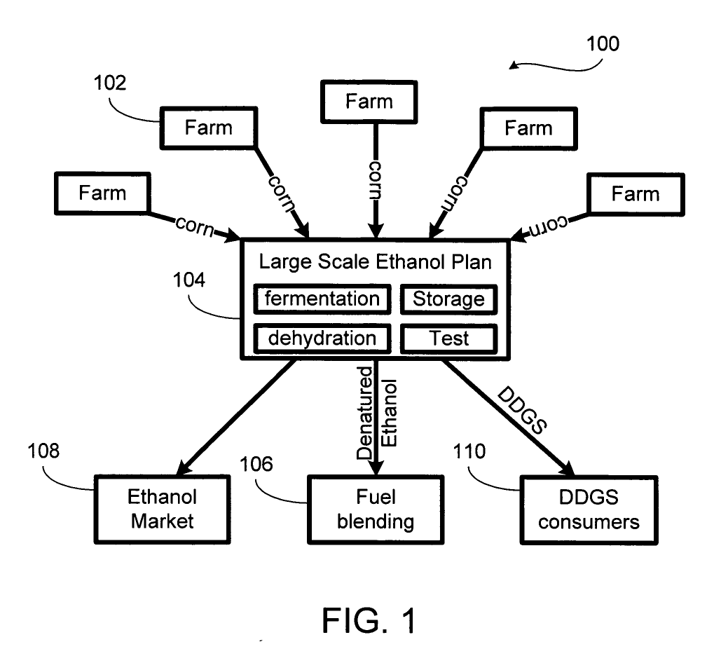

FIG. 2 illustrates an updated market process 200 according to the present invention.

FIG. 3 is a flow chart for the updated market process according to the invention.

### DETAILED DESCRIPTION OF THE INVENTION

The present invention is a system and method that provides a dehydration service to those who have hydrous alcohol and desire absolute alcohol, typically for mixing with gasoline to create motor fuel. The present invention is different from the prevailing designs and models. The present invention preferably utilizes a portable, skid-based dehydration facility that can be mass produced in a factory and shipped to a location to dehydrate ethanol. The system operates in a batch process that does not interrupt upstream operations and is specifically designed to handle small batches of farm distilled ethanol. The present system and method therefore allows the blending of the absolute ethanol at or near the farmer(s) with gasoline and delivery to nearby E-85 stations or fuel distribution centers. Alternatively, the blended fuel may also be used by the farmer himself.

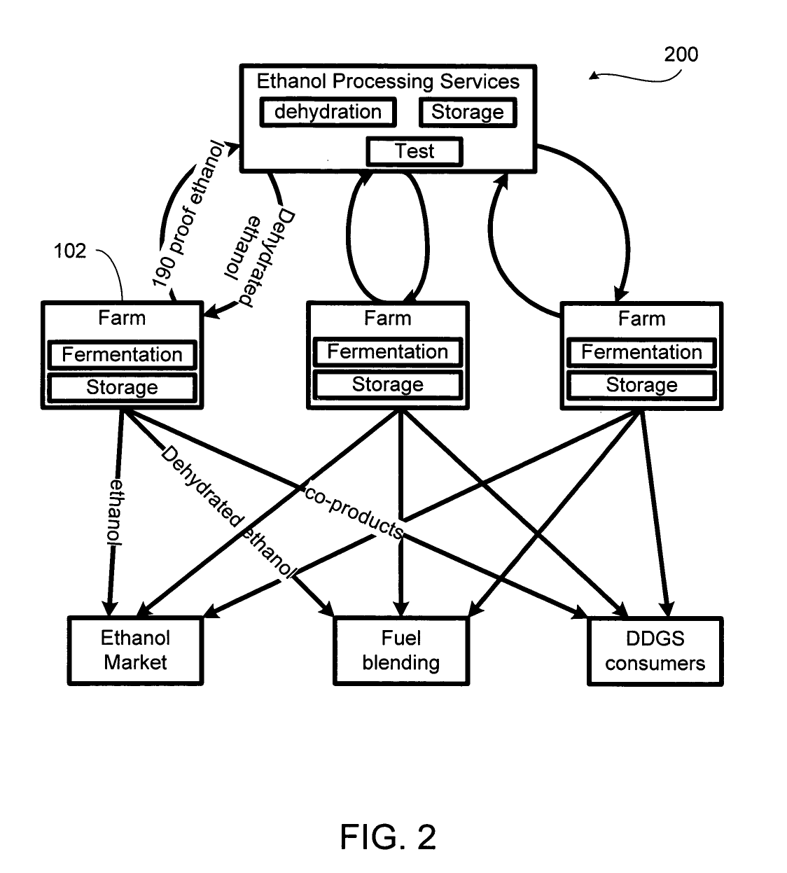

FIG. 2 illustrates an updated market process 200 according to the present invention. In this updated market process 200, the farmers (here, corn producers) can take advantages of improvements done on equipments and incorporate some aspect of ethanol production. After some on-site construction to install necessary alcohol producing equipment, a farmer, instead of selling corn to a larger ethanol production plant, may grind corn, add water to ground corn for fermentation, and produce hydrous ethanol at his farm through distillation. The farmer may operate the alcohol producing equipment himself or hire a third party to operate the alcohol producing equipment for him. After the fermentation and distillation, the farmer may then contract an ethanol processing service provider to dehydrate ethanol, i.e., to remove water from the ethanol he has produced. After the water is removed from the ethanol, the dehydrated ethanol is returned to the farmer, who can then sell the dehydrated ethanol to fuel blending companies, thus deriving a bigger profit. Also in this process, the farmer will be able to obtain additional financial benefits by selling or using byproducts, such as distillers dried grains with solubles (DDGS), or wet distillate grains (WDG) and thin stillage directly to a byproduct market. The farmer may also sell the un-treated ethanol or denatured ethanol directly at the ethanol market thus further enhancing his profits. The denaturing of ethanol is generally accomplished by adding gasoline, denatonium benzoate, or other denaturant. Alternatively, the farmer may blend the dehydrated ethanol with gasoline to produce E-85 fuel and then sell the E-85 fuel to a proper fuel station. In yet another alternative, the farmer may simply distill ethanol and store it for pick up by buyers.

During the alcohol production process, the farmer may test the product from each stage to ensure the quality and fitness of the product for the next production stage. For example, the farmer may test denatured alcohol according to ASTM D 4806 Standard Specification for Denatured Fuel Ethanol for Blending with Gasoline for Use as Automotive Spark Ignition Engine Fuel. The farmer may also test the dehydrated ethanol produced by the ethanol processing service provider. The alcohol production process may be adjusted to correct any problem observed from the testing results. Testing may also be done at the ethanol processing service provider's facility. The blended fuel may be tested according to ASTM D 5798.

The ethanol processing service provider may establish a small or large processing plant near where the corn (or any other alcohol production suitable farm product, such as barley, rye, sorghum, sugar cane, potatoes, sugar beets, and wheat) producers are located. Alternatively, a transportable processing facility may also be employed, i.e., the transportable processing facility is transported to a location near the corn producers. The ethanol processing service provider can then provide services of ethanol dehydration to the corn producers. The services provided are similar to the services executed by a traditional large ethanol plant except for corn grinding and fermentation. The services may be rendered based on the volume of ethanol to be dehydrated or any other type of fee arrangement. The ethanol processing service provider may also buy the ethanol produced from the corn instead of providing only the dehydration services. The ethanol processing service provider will then dehydrate the ethanol and sell the ethanol to fuel blending companies.

In the updated market process 200, a corn producer will have more control of farm products he produces and consequently derive more financial benefits. The farmer can disperse thin stillage as fertilizer on his land, thus saving money and reducing use of chemical fertilizers. The farmer can also use wet distillers grains (WDG) for feed with farm animals he may have, thus further saving money. The updated market process 200 according to the invention can also generate new business opportunities to third parties. For example, new distilling equipments can be sold to the corn producers and equipment maintenance services will be needed for these equipments. Local transportation of hydrous ethanol to and from the ethanol processing service provider will also be required. Molecular sieves or other dehydrating equipment will be needed by the ethanol processing service providers.

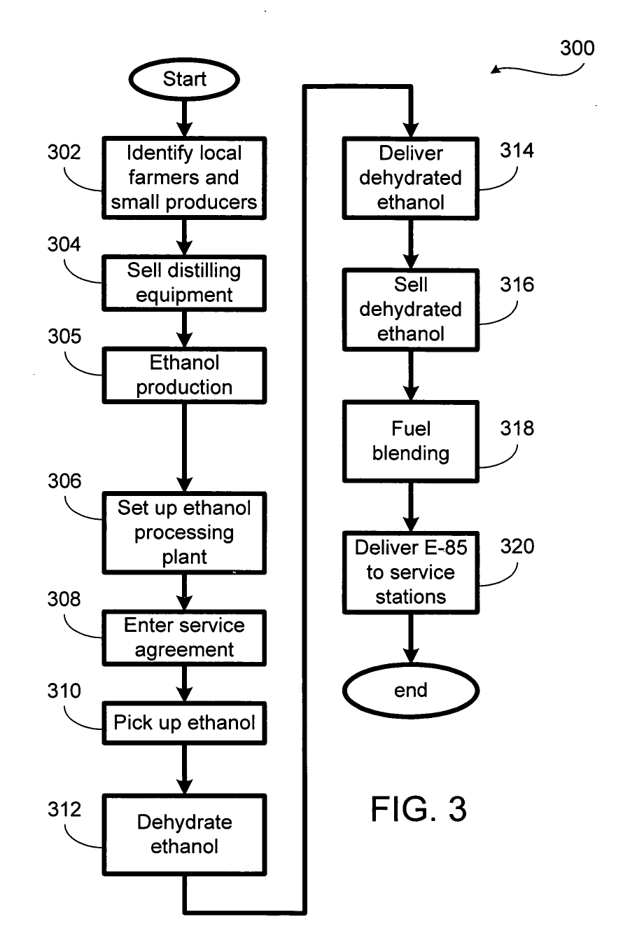

The updated market process 200 provides a system and method that utilize a portable ethanol dehydration system, such as one or more portable molecular sieves, to provide parties, particularly farmers, who have hydrous ethanol the ability to dehydrate the ethanol for ultimate blending with gasoline to become a motor fuel or for marketing directly to the ethanol market. FIG. 3 is a flow chart for the updated market process 300 according to the invention. Initially, local farmers or other small producers interested in making ethanol as a value added step to growing a carbohydrate source are identified, step 302. The list of interested farmers can then be provided to equipment sellers, who can then sell the equipments, step 304, and help the farmers to establish an ethanol production facility. After the ethanol production facility is set up, the farmers can then produce ethanol on their farms, step 305.

The ethanol processing service providers can set up facilities adjacent to the farmers or on the farmers' site, step 306, and sell their services to the farmers, step 308. The ethanol processing service providers will test ethanol for quality and then pick up ethanol from each farm, step 310. Alternatively, the farmers may also deliver the ethanol to the ethanol processing service providers. The ethanol is processed at a local ethanol processing service facility and water is then removed from the hydrous ethanol, step 312. After dehydration, the dehydrated ethanol is returned to the farmer, step 314, who can then sell the dehydrated ethanol directly to a fuel blending facility, step 316. The farmer may test the dehydrated ethanol for quality purposes. Alternatively, the ethanol processing service provider may also provide fuel blending services. The ethanol processing service provider may buy the hydrous ethanol from the farmer, dehydrate the hydrous ethanol, blend the dehydrated ethanol with gasoline to the desired concentration, step 318, which normally results in E-85 grade fuel, and sells the E-85 fuel to a proper refueling station, step 320.

While the present invention might not be as efficient in dehydration as the larger plants, it can be picked up and moved to another location depending on local farmer participation. This allows the farmers who distill their own ethanol to be customers, and not competitors. Further, raw material suppliers will also transfer some value added steps to farmers that are far better able to use the fermentation byproducts as feed or fertilizer.

Although preferred embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. Moreover, although specific terms are employed herein, they are used in a generic and descriptive sense only, and not for the purposes of limiting the described invention.

What is claimed is:

1. A method for obtaining dehydrated alcohol by farm product producers who produce ethanol-production-capable farm products, the method comprising the steps of:
producing hydrous ethanol;
delivering the hydrous ethanol to a dehydration service provider; and
receiving dehydrated ethanol from the dehydration service provider.

2. The method of claim 1, further comprising the step of entering a service agreement with the dehydration service provider.

3. The method of claim 1, further comprising the step of selling the dehydrated ethanol to a fuel blending company.

4. The method of claim 1, further comprising the step of selling the hydrous ethanol.

5. The method of claim 1, further comprising the step of blending the dehydrated ethanol with gasoline to produce E-85 fuel.

6. The method of claim 1, further comprising the step of selling ethanol-production-capable farm products in addition to hydrous alcohol.

7. The method of claim 1, further comprising the step of denaturing the hydrous ethanol.

8. The method of claim 1, further comprising the step of testing dehydrated ethanol for quality.

9. The method of claim 1, further comprising the steps of:
installing alcohol producing equipment in a farm; and
operating the alcohol producing equipment by a third party.

10. A method for providing ethanol dehydration services to farm product producers who produce hydrous ethanol, the method comprising the steps of:
receiving hydrous ethanol from a farm product producer;
dehydrating the hydrous ethanol; and
returning dehydrated ethanol to the farm product producer.

11. The method of claim 10, further comprising the step of entering a service agreement with the farm product producer.

12. The method of claim 10, further comprising the step of producing a blended fuel by blending the dehydrated ethanol with gasoline.

13. The method of claim 12, further comprising the step of selling the blended fuel.

14. A method for providing ethanol dehydration services to farm product producers who produce hydrous ethanol, the method comprising:
a step for receiving hydrous ethanol from a farm product producer;
a step for dehydrating the hydrous ethanol; and
a step for returning dehydrated ethanol to the farm product producer.

15. The method of claim 14, further comprising a step for entering a service agreement with the farm product producer.

16. The method of claim 14, further comprising a step for producing a blended fuel by blending the dehydrated ethanol with gasoline.

17. The method of claim 16, further comprising a step for selling the blended fuel.
